(19) United States
(12) Patent Application Publication
(10) Pub. No.: US 2025/0255598 A1
(43) Pub. Date: Aug. 14, 2025

(54) ENDOSCOPIC ULTRASOUND-GUIDED ANCHOR DEVICE FOR TRANSLUMINAL APPOSITION

(71) Applicant: EndoFix Medical Technologies, Inc., Plainville, MA (US)

(72) Inventors: Rickey Hart, Wrentham, MA (US); William Blenkhorn, Medway, MA (US)

(21) Appl. No.: 19/054,593

(22) Filed: Feb. 14, 2025

Related U.S. Application Data
(60) Provisional application No. 63/553,271, filed on Feb. 14, 2024.

(51) Int. Cl. A61B 17/04

(52) U.S. Cl. CPC A61B 17/0401; A61B 2017/0409

(57) ABSTRACT

In illustrative embodiments, an anchor device comprising an anchor assembly and a locking device connected via a single flexible strand fixes a stomach wall to an organ wall so that a stent may be positioned between the stomach (or duodenum) and the organ, such as a small intestine, or a gallbladder. An anchor assembly is implanted through the stomach wall into a wall of an internal organ for fixation of the stomach wall to the internal organ wall. Fixing the two organs together with the anchor device at the correct location prior to puncturing an opening between the two organs increases the ease and accuracy of making the incision in each of the organ walls. Fixation of the two organ walls with of the anchor device also simplifies the placement and deployment of the stent by reducing the shifting and movement of the organ walls relative to each other.

### PRIORITY

This patent application claims priority from provisional U.S. patent application No. 63/553,271, filed Feb. 14, 2024, entitled, “ENDOSCOPIC ULTRASOUND-GUIDED ANCHOR DEVICE FOR TRANSLUMINAL APPOSITION,” and naming Rickey Hart as inventor, the disclosure of which is incorporated herein, in its entirety, by reference.

### FIELD

Illustrative embodiments of the invention generally relate to devices and methods for endoscopic surgery and, more particularly, various embodiments of the invention relate to anchor devices.

### BACKGROUND

Endoscopic ultrasound-guided gastrojejunostomy (EUS-GJ) and endoscopic ultrasound-guided gallbladder drainage (EUS-GBD) are minimally invasive procedures that create a bypass between the stomach and small intestine (EUS-GJ), and inserting a stent into the gallbladder through the wall of the small intestine or stomach (EUS-GBD), respectively. These procedures are used to treat gastric outlet obstruction caused by tumors, chronic diseases, etc. (EUS-GJ), as well as treating gallbladder infections and obstructions (EUS-GBD).

These procedures involve inserting a stent between the stomach (or duodenum) and into the wall of the small intestine, or into the gallbladder. A significant challenge to the process of installing the stent between the stomach and the other end of the stent is locating the target of the puncture in the small intestine or gall bladder. Another challenge is holding (e.g., fixing) the second organ (the small intestine or the gall bladder) in place while the puncture is made and the stent is placed. There is a particular need in the medical procedures of endoscopic ultrasound-guided gastrojejunostomy and endoscopic ultrasound-guided gallbladder drainage for fixing the two organs together at the correct location prior to puncturing the opening between the organs and placing the stent.

### SUMMARY OF VARIOUS EMBODIMENTS

In accordance with one embodiment of the invention, an anchor assembly for fixation of a stomach wall to an internal organ wall includes a flexible strand; and a plurality of braided anchor segments slidably coupled to the flexible strand. The flexible strand passes through a bore hole that traverses from a proximal end to a distal end of each of the plurality of braided anchor segments such that the proximal end of a first braided anchor segment is adjacent to a distal end of a second braided anchor segment. The plurality of braided anchor segments are cinched together forming an anchor assembly to provide tissue fixation.

Each of the plurality of braided anchor segments may include straight braided suture segments that have been deformed by application of tension on the flexible strand. The flexible strand may be a monofilament. The flexible strand may be a suture. The suture may be a braided polyester suture.

Each of the plurality of braided anchor segments may be standard braided polyester suture that is cut into predetermined lengths.

The cinched plurality of braided anchor segments may have a purse string effect on the plurality of braided anchor segments such that the plurality of flexible braided anchor segments is drawn together to provide tissue fixation.

In accordance with another embodiment of the invention, a method for implanting anchor assembly connected via a single flexible strand for fixation of a stomach wall to an internal organ wall includes puncturing an inside surface of the stomach wall tissue at a first location with a delivery needle, puncturing an outside surface of the internal organ wall at a second location with the delivery needle, and extruding an anchor assembly from the delivery needle.

The anchor assembly includes the single flexible strand, and a plurality of braided anchor segments slidably coupled to the single flexible strand. The single flexible strand passes through a bore hole that traverses from a proximal end to a distal end of each of the plurality of braided anchor segments such that the proximal end of a first braided anchor segment is adjacent to a distal end of a second braided anchor segment. Pulling the flexible strand a first time to deform the plurality of braided anchor segments forms the anchor assembly. The anchor assembly is configured to anchor an outside surface of the stomach wall to the outside surface of the internal organ wall, and prevent the anchor assembly from being pulled thru internal organ wall to the stomach wall.

The method further includes withdrawing the delivery needle from the stomach wall and the internal organ wall, securing the flexible strand to keep the stomach wall anchored to the internal organ wall fixated by the anchor assembly, and cutting the flexible strand. The flexible strand is cut a distance away from the proximal end of a final braided anchor segment of the plurality of the braided anchor segments.

The extruding the anchor assembly may be accomplished by advancing a drive tube to push the plurality of braided anchor segments out of the delivery needle.

The securing the flexible strand may include locking the flexible strand in place using a locking mechanism including at least one of a tag, a barb, a cinch, a toggled needle, or a distel knot. The securing the flexible strand may include a locking device or an overhand knot that can be run down to a fixation site.

Puncturing the outside surface of the internal organ wall at the second location with the delivery needle may follow continuously from puncturing the inside surface of the stomach wall tissue at the first location with the same delivery needle. The stomach may include a duodenum.

In accordance with another embodiment of the invention, a surgical device for implanting a flexible braided anchor assembly for affixing a stomach wall to an internal organ wall includes a handle assembly. The handle assembly has a cylindrical bore passing through the handle assembly on a center axis along a length of the handle assembly.

The surgical device further includes an outer tube positioned in the cylindrical bore of the handle assembly. The outer tube is hollow and has a common center line with the cylindrical bore of the handle assembly. The outer tube includes a delivery needle having a needle tip at a distal end of the outer tube.

The surgical device further includes a plurality of braided suture segments slidably coupled to a flexible strand. The flexible strand is translationally threaded through plurality of braided suture segments, and the plurality of braided suture segments are positioned in the outer tube.

The surgical device further includes a first drive tube positioned in the outer tube and having a common center line with the outer tube. The first drive tube is configured to extrude the braided suture segments.

Each of the plurality of braided suture segments may be a hollow braided suture segment. The suture may be a monofilament strand, and the plurality of braided suture segments may be a plurality of hollow braided suture segments aligned end to end having the monofilament strand threaded through the aligned plurality of hollow braided suture segments.

A column strength of the plurality of hollow braided suture segments may be increased by application of at least one of heat or a cyanoacrylate coating to each of the segments of the plurality of hollow braided suture segments.

A braided suture segment of the plurality of braided suture segments may have a length of between about 5 mm and about 50 mm. Each of the braided suture segments of the plurality of braided suture segments may a length of between about 5 mm and about 12 mm.

A distal end of the flexible strand threaded thru the first braided suture segment may be looped around the first braided suture segment and knotted around the flexible strand between the first braided suture segment and the first drive tube.

Each of the plurality of braided suture segments may be treated to increase a rigidity of the plurality of braided suture segments slidably coupled to the flexible strand. The treatment may include heating each of the plurality of braided suture segments, or applying a cyanoacrylate coating to each of the plurality of braided suture segments.

In accordance with another embodiment of the invention, a method for implanting an anchor assembly connected via a single flexible strand through a drain tube placed from the outside of a body for fixation of a gall bladder wall to a stomach wall includes puncturing an inside surface of the gall bladder wall tissue at a first location with a delivery needle. The delivery needle is brought into the interior of the gall bladder through a drain tube placed between outside of the body and the gall bladder.

The method further includes puncturing an outside surface of the stomach wall at a second location with the delivery needle, and extruding an anchor assembly from the delivery needle. The anchor assembly includes the single flexible strand, and a plurality of braided anchor segments slidably coupled to the single flexible strand. The single flexible strand passes through a bore hole that traverses from a proximal end to a distal end of each of the plurality of braided anchor segments such that the proximal end of a first braided anchor segment is adjacent to a distal end of a second braided anchor segment.

The method further includes pulling the flexible strand a first time to deform the plurality of braided anchor segments forming the anchor assembly. The anchor assembly is configured to anchor an inside surface of the stomach wall to the outside surface of the gall bladder wall, and prevent the anchor assembly from being pulled thru the stomach wall to the gall bladder wall.

The method further includes withdrawing the delivery needle from the stomach wall and the gall bladder wall, securing the flexible strand to keep the stomach wall anchored to the gall bladder wall fixated by the anchor assembly, and cutting the flexible strand. The flexible strand is cut a distance away from the proximal end of a final braided anchor segment of the plurality of the braided anchor segments.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1A schematically shows a portion of a procedure for fixing one organ to another according to one embodiment of the present disclosure.

FIG. 1B schematically shows another portion of a procedure for fixing one organ to another according to one embodiment of the present disclosure.

FIG. 1C schematically shows another portion of a procedure for fixing one organ to another according to one embodiment of the present disclosure.

FIG. 1D shows an expansion of the indicated region of the schematic drawing of FIG. 1C.

FIG. 2 shows a schematic drawing illustrating an embodiment of an endoscopic ultrasound-guided gastrojejunostomy according to one embodiment of the present disclosure.

FIG. 3 shows a schematic drawing illustrating an embodiment of an endoscopic ultrasound-guided gallbladder drainage according to one embodiment of the present disclosure.

FIG. 4A shows a schematic cross section illustration of a delivery device with a delivery needle having a needle tip according to one embodiment of the present disclosure.

FIG. 4B shows a schematic cross section illustration of a plurality of braided suture segments after being extruded from the needle tip according to one embodiment of the present disclosure.

FIG. 4C shows a schematic cross section illustration of the flexible braided suture segments after the suture has been pulled taut according to one embodiment of the present disclosure.

FIG. 4D shows a schematic cross section illustration of the flexible braided suture segments and the suture after the delivery device has been pulled back according to one embodiment of the present disclosure.

FIG. 4E shows a schematic cross section illustration of the anchor assembly fixed to the organ wall and the stomach wall according to one embodiment of the present disclosure.

FIG. 5A shows a top view of the delivery device according to one embodiment of the present disclosure.

FIG. 5B shows a side view of the delivery device according to one embodiment of the present disclosure.

FIG. 6A shows an angled top view of the delivery device with the safety pull in position on the delivery device according to one embodiment of the present disclosure.

FIG. 6B shows an angled top view of the delivery device with the safety pull removed from its position on the delivery device according to one embodiment of the present disclosure.

FIG. 6C shows an angled top view of the delivery device with the safety pull removed and pusher 64 having been advanced toward the front handle according to one embodiment of the present disclosure.

FIG. 7A shows a schematic cross section illustration of the flexible braided anchor segments located in the delivery needle which is concentrically positioned in the outer tube according to one embodiment of the present disclosure.

FIG. 7B shows a schematic cross section illustration of an expansion of FIG. 7A according to one embodiment of the present disclosure.

FIG. 8 shows a top view of the delivery device with the delivery needle extended according to one embodiment of the present disclosure.

FIG. 9A shows a schematic drawing of the plurality of braided suture segments with the suture looping back to be formed in a slip knot according to one embodiment of the present disclosure.

FIG. 9B shows a schematic drawing of the plurality of braided suture segments after the suture has been pulled and the plurality of braided suture segments has been deformed into the anchor assembly according to one embodiment of the present disclosure.

FIG. 10A shows a schematic illustration of an embodiment of a long braided suture segment on a suture according to one embodiment of the present disclosure.

FIG. 10B shows a schematic illustration of a long braided anchor assembly) segment as it is deployed on the distal side of a tissue according to one embodiment of the present disclosure.

FIG. 11 shows steps of an embodiment of a method for implanting braided anchor segments connected via a single flexible strand for tissue fixation according to one embodiment of the present disclosure.

### DESCRIPTION OF ILLUSTRATIVE EMBODIMENTS

In illustrative embodiments, an anchor device comprising an anchor assembly and a locking device connected via a single flexible strand fixes a stomach wall to an organ wall so that a stent may be positioned between the stomach (or duodenum) and the organ, such as a small intestine, or a gallbladder. An anchor assembly is implanted through the stomach wall into a wall of an internal organ for fixation of the stomach wall to the internal organ wall. Fixing the two organs together with the anchor device at the correct location prior to puncturing an opening between the two organs increases the ease and accuracy of making the incision in each of the organ walls. Fixation of the two organ walls with of the anchor device also simplifies the placement and deployment of the stent by reducing the shifting and movement of the organ walls relative to each other. The anchor assembly comprises at least one braided suture segment, or a plurality of braided anchor segments that is implanted using a delivery device with a handle assembly and is positioned and controlled through an endoscope. The anchor assembly, formed of at least one braided suture segment, is extruded through a needle tip at the distal end of a delivery needle. Details of illustrative embodiments are discussed below.

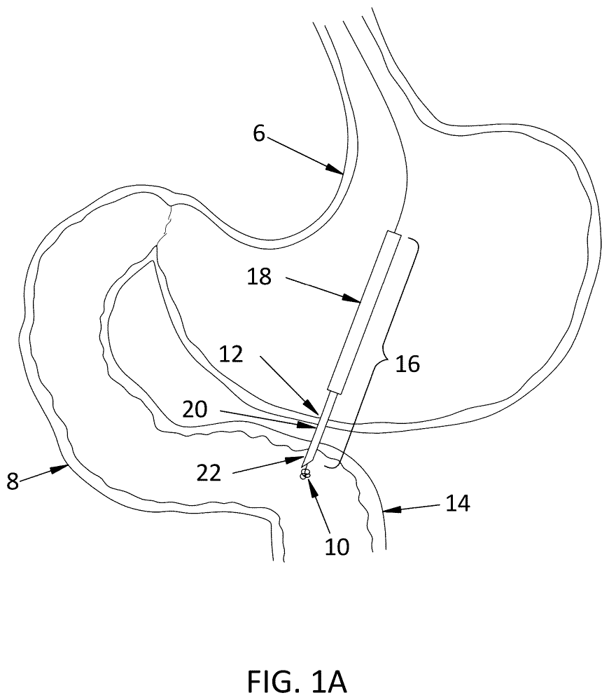

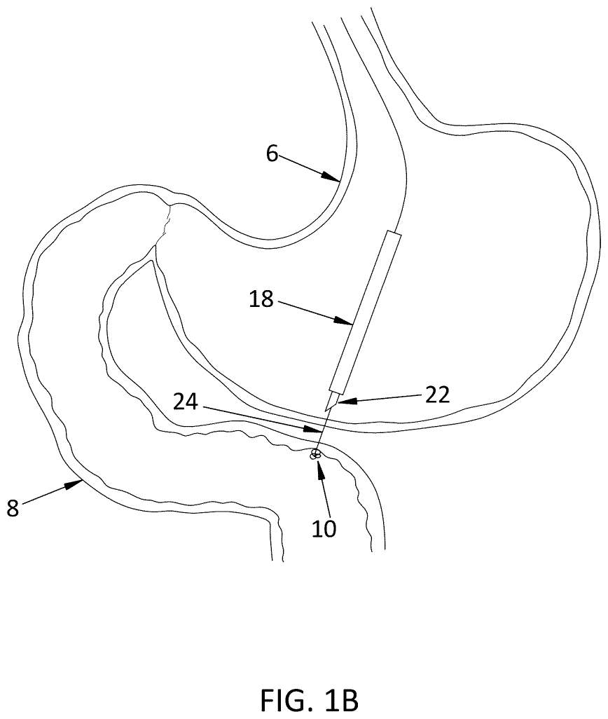

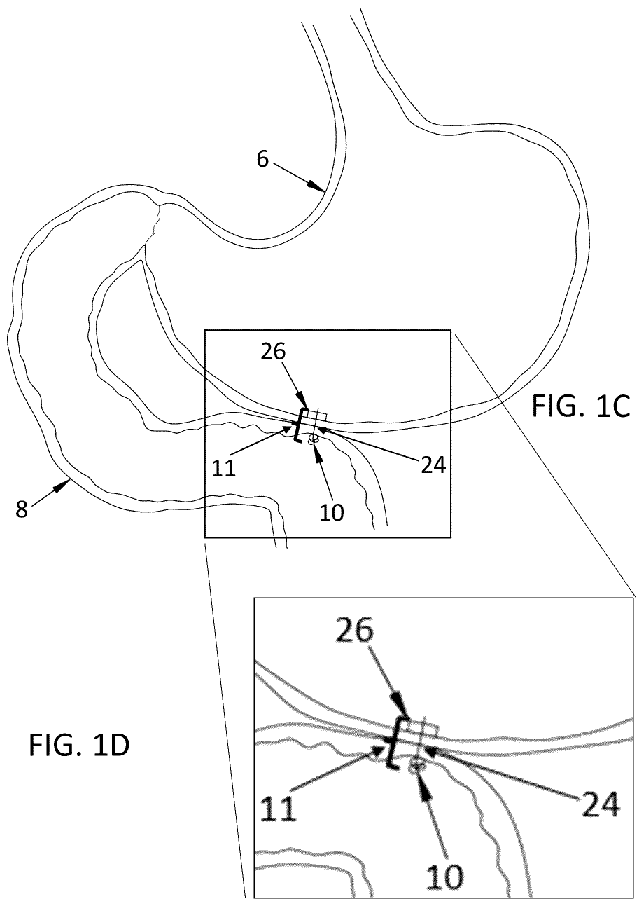

FIG. 1A, FIG. 1B, and FIG. 1C schematically illustrate fixing a stomach 6 to a small intestine 8 prior to placing a stent between the stomach 6 and the small intestine 8 as an initial step for minimally invasive endoscopic ultrasound-guided gastrojejunostomy (EUS-GJ) procedure that creates a bypass between the stomach and small intestine.

As shown in FIG. 1A, the EUS-GJ procedure begins with implanting an anchor assembly 10, comprising a flexible braided anchor assembly, for affixing the stomach wall 12 to the small intestine wall 14. An incision is made on the inside of the stomach wall 12 with a delivery device 16 having a handle assembly 18 including a delivery needle 20 with a needle tip 22 at the distal end of the delivery needle 20.

The needle tip 22 is then pushed into the outside surface of the small intestine 14 to puncture the small intestine 8. In some embodiments, the needle tip 22 is pushed continuously through the inside surface of the stomach 12 and into the outside surface of the small intestine 14. In some embodiments, once the needle tip 22 is pushed through the inside surface of the stomach 12, the delivery device 16 is repositioned under ultrasound guidance, and the needle tip 22 is pushed and into the outside surface of the small intestine 14. Once the needle tip 22 is inside of the small intestine 8, the anchor assembly 10 may be extruded from the needle tip 22. After extrusion, the anchor assembly 10, comprised of a flexible braided anchor assembly and a flexible strand 24 (e.g., a suture), is positioned inside the small intestine 8.

Once positioned in the small intestine 8, the flexible strand 24 is pulled a first time to deform the plurality of braided anchor segments forming the anchor assembly 10. The anchor assembly is configured to anchor an outside surface of the stomach wall to the outside surface of the small intestine wall, and to prevent the anchor assembly 10 from being pulled thru small intestine wall 14 to the stomach wall 12.

As shown in FIG. 1B, once the braided anchor segments are extruded into the small intestine and the have been deformed into the anchor assembly 10, the delivery device 16 is retracted from the small intestine 8 and the stomach wall 12. The suture 24 is pulled and the small intestine 8 is drawn toward the stomach wall 12. Once the small intestine wall 14 is drawn toward the stomach wall 12, the suture may be secured and cut.

FIG. 1C shows a schematic drawing of the anchor device 11 fixing the stomach 6 to the small intestine 8. Now the delivery device can be removed and a locking device 26 (e.g., locking mechanism) such as a cinch or special knot can be pushed down to complete and lock the fixation. Additional locking mechanisms that may be used include a tag, a barb, a cinch, a toggled needle, or distal knot. The anchor assembly 10 may be secured against the inside surface of the small intestine by pulling the suture 24 tight and attaching a locking device 26 to the suture 24 to keep the tension in the suture 24. Once the locking device 26 is secured in position, the suture 24 is cut to a predetermined length and the anchor device 11 is in position.

FIG. 1D shows an expansion of the indicated region of the schematic drawing of FIG. 1C. The anchor device 11 fixing the stomach wall 6 to the intestine wall 8 is shown as comprising the anchor assembly 10, the suture 24, and the locking device 26.

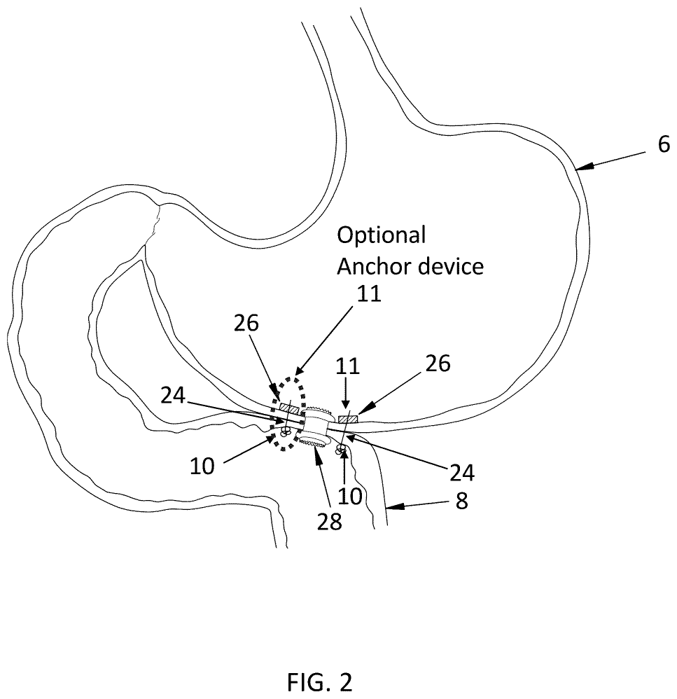

FIG. 2 shows a schematic drawing illustrating an embodiment of an endoscopic ultrasound-guided gastrojejunostomy (EUS-GJ), a minimally invasive procedure that creates a bypass between the stomach 6 and the small intestine 8. In embodiments, the EUS-GJ procedure may include implanting one or more anchor devices 11 prior to placing the stent 28. In some embodiments, more than one anchor device 11 may be placed to secure the tissues. FIG. 2 illustrates how the placement of a second anchor device 11 is optional. The stent 28 has a flange on each end of the stent 28 to lock the stent 28 in place between the flexible stomach wall 14 and small intestine wall 14.

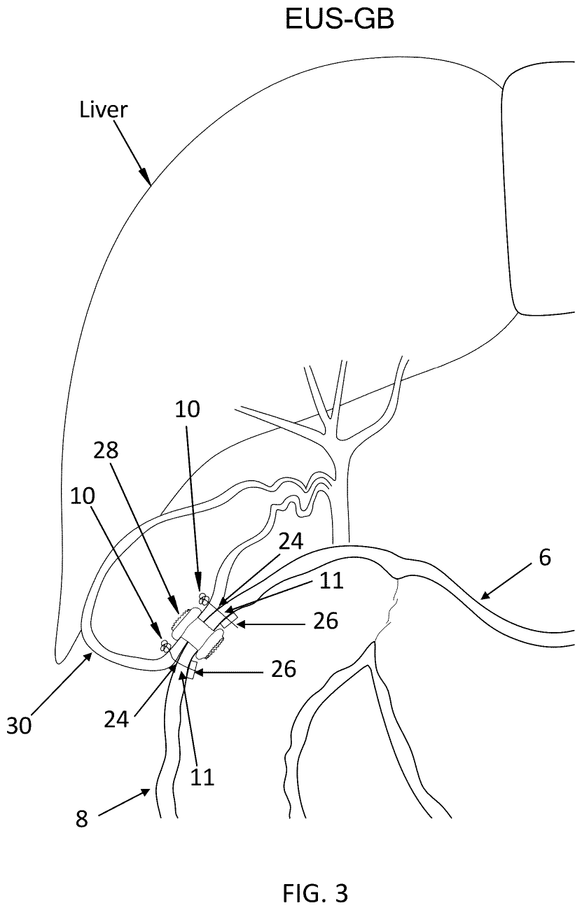

FIG. 3 shows a schematic drawing illustrating an embodiment of an endoscopic ultrasound-guided gallbladder drainage (EUS-GBD), a minimally invasive procedure that creates a bypass between the stomach and the gall bladder. In embodiments, the EUS-GB procedure may include implanting one or more anchors prior to placing the stent 28. The stent 28 has a flange on each end of the stent 28 to lock the stent 28 in place between the flexible stomach wall 14 and gall bladder wall 30.

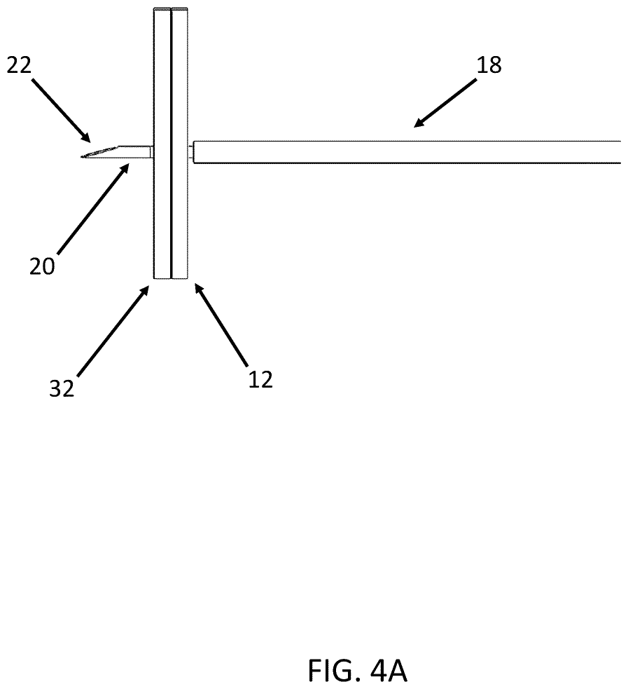

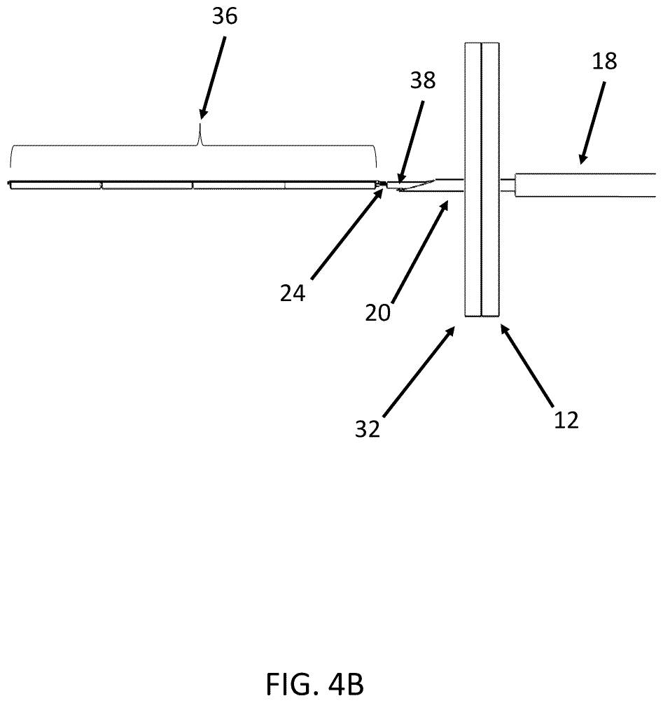

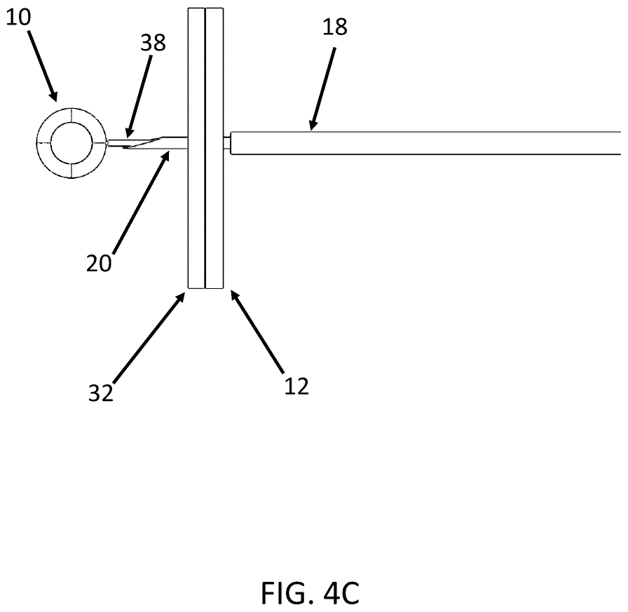

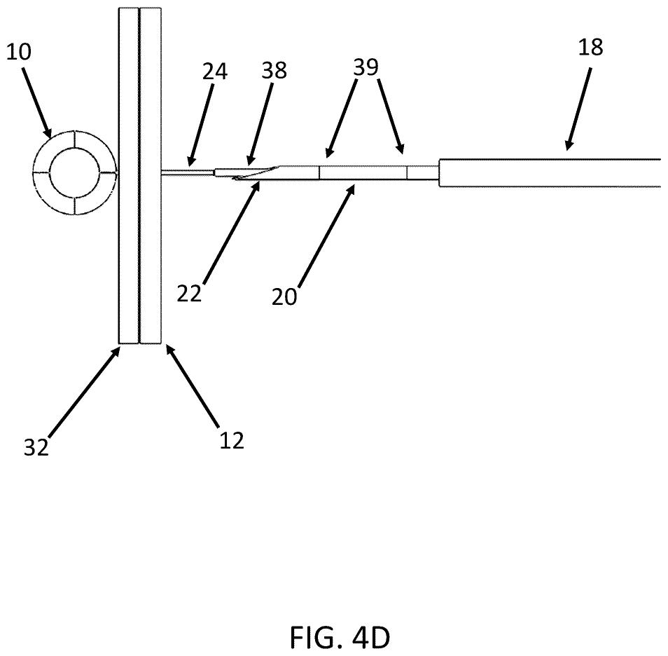

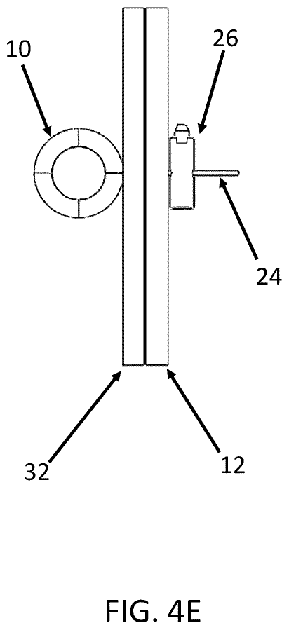

FIGS. 4A-4E show simplified schematic cross section illustrations of some embodiments of delivery devices deploying an assembly of braided anchor segments. For clarity of illustration, the figures FIGS. 4A-4E have been simplified and may not necessarily show every detail of the delivery devices.

FIG. 4A shows a simplified schematic cross section illustration of a handle assembly 18 with a delivery needle 20 having a needle tip 22 after puncturing a stomach wall 12 and an organ wall 32 according to an embodiment of the present disclosure. In some embodiments, the organ wall 32 may be a small intestine wall 14, a gall bladder wall 30, or a wall of another organ 32. The needle 22 punctures a first hole in the stomach wall 12 and then the organ wall 32. The needle 22 is at a distal end of an outer tube, and the outer tube is coupled to the handle assembly 18.

In some embodiments, the needle 22 and the outer tube may be fabricated as a single piece, whereas in other embodiments, the needle 22 and the outer tube may be assembled from separate pieces. The needle 22 and the outer tube may be fabricated from one or more rigid materials, such as a steel, aluminum, titanium, and so on. The rigid material may also include metallic alloys such as nitinol. In embodiments, when the needle 22 and the outer tube are assembled from separate pieces, the needle 22 and the outer tube may be fabricated from different rigid materials.

FIG. 4B shows a simplified schematic cross section illustration of a plurality of braided suture segments 36 (e.g., a flexible braided anchor assembly) after being extruded from the needle 20 on the distal side of the an organ wall 32. The plurality of braided suture segments 36 is pushed out distally out of the needle 20 by a drive tube 38. The flexible strand (e.g., suture) 24 is shown threaded thru the flexible braided suture segments 36, looped around, and knotted with a slidable slip knot around the suture between proximal braided suture segment and the drive tube 38. In some embodiments, at least one braided suture segment may be used in place of a plurality of braided suture segments.

In embodiments, the flexible strand (e.g., suture) 24 may be a monofilament. In other embodiments, the flexible strand 24 may be a suture. The suture 24 may be a braided polyester suture. In embodiments, the suture 24 may be a FiberWire®, TigerWire®, or FiberChain® suture, although any type of suture 24 may be utilized, including cored or coreless sutures. In another embodiment, the flexible strand 24 may be suture tape, such as FiberTape®. The flexible strand 24 may include any soft, flexible strand of material.

In some embodiments, the braided suture segments 36 may be strengthened by heating the braided suture segments 36. The braided suture segments 36 may also be strengthened by coating them with a cyanoacrylate coating.

FIG. 4C shows a simplified schematic cross section illustration of the flexible braided suture segments 36 after the suture 24 has been pulled taut a first time. As a result of the suture 24 being pulled tight, the flexible braided suture segments 36 has been deformed into an anchor assembly 10. In some embodiments, the anchor assembly 10 may have circular shapes, while in other embodiments the anchor assembly may have a irregular shapes.

FIG. 4D shows a simplified schematic cross section illustration of the anchor assembly 10 and the suture 24 after the handle assembly 18 with the delivery needle 20 and the needle tip 22 have been pulled back and retracted from the organ wall 32 and the stomach wall 12. By pulling the suture 24, the slip knot is tightened to secure the anchor assembly 10 on the distal surface of the organ wall 32. Depth line marks 39 on the needle 20 are used to gauge the length of needle extending out of the handle assembly 18.

FIG. 4E shows a simplified schematic cross section illustration of the anchor assembly 10 (e.g., deformed plurality of flexible braided suture segments) fixed to the organ wall 32 and the stomach wall 12. The anchor assembly 10 is secured by locking the suture 24 in place with a locking device 26. In embodiments, the anchor assembly 10 may be secured in place with any one of locking mechanisms including a locking device 26, a tag, a barb, a cinch, a toggled needle, or a distel knot.

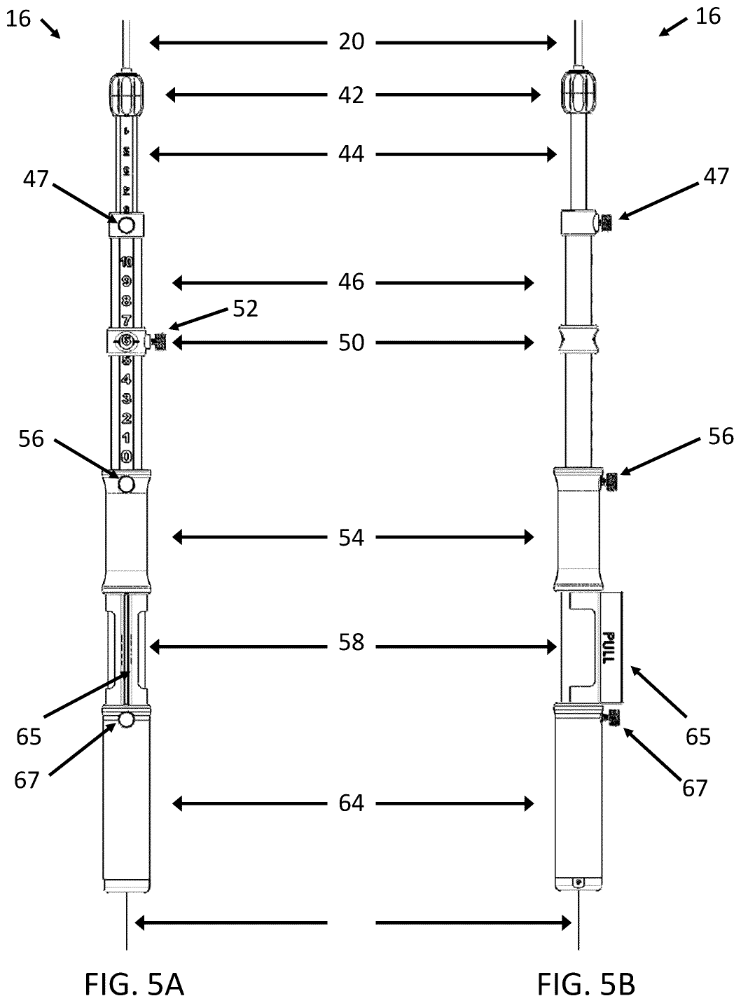

FIG. 5A shows a top view of the delivery device 16, and FIG. 5B shows a side view of the delivery device 16. The delivery device 16 includes a delivery needle 20 positioned concentrically in a scope attachment 42 and a scope adjustment shaft 44. The scope attachment shaft 44 is concentrically positioned in a shaft 46 with a shaft adjustment thumb screw 47. A slide indicator 50 with a slide indicator thumb screw 52 is slidably mounted on the shaft 46. The slide indicator 50 allows for the length of the needle 20 that extends beyond the handle assembly to be controlled. The depth line marks 39 can be used with the slide indicator 50 to index the extension length of the needle 20. The shaft 46 is concentrically mounted in a front handle 54. The front handle 54 has a front handle thumb screw 56. A rear handle 58 with a end cap is concentrically mounted between the front handle 54 and a pusher 64. A safety pull 65 is removably mounted on the rear handle 58 between the front handle 54 and a pusher 64. The pusher 64 includes a pusher thumb screw 67. The flexible strand (e.g., suture) is threaded concentrically through the entire delivery device 16.

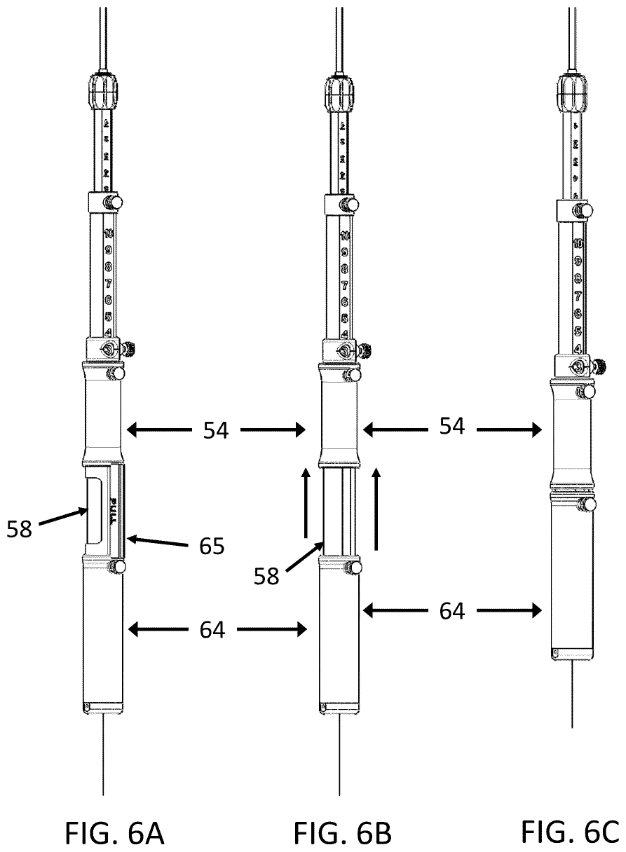

FIGS. 6A through 6C illustrate how the delivery device 16 is mechanically operated to extrude the a flexible braided anchor assembly 36. FIG. 6A shows an angled top view of the delivery device 16 with the safety pull 65 in position on the rear handle 58 between the front handle 54 and a pusher 64.

FIG. 6B shows an angled top view of the delivery device 16 with the safety pull 65 removed from the position on the rear handle 58 between the front handle 54 and a pusher 64. The arrows indicate a direction that the pusher 64 is moved to push the flexible anchor assembly 36 out of the needle point 22.

FIG. 6C shows an angled top view of the delivery device 16 with the safety pull 65 removed and pusher 64 having been advanced toward the front handle 54. After removing the safety pull 65, the pusher 64 may be moved forward, as indicated by the direction arrows next to rear handle 58. Moving the pusher forward engages an inner drive tube that pushes the flexible braided anchor assembly 36 out of the delivery needle 20.

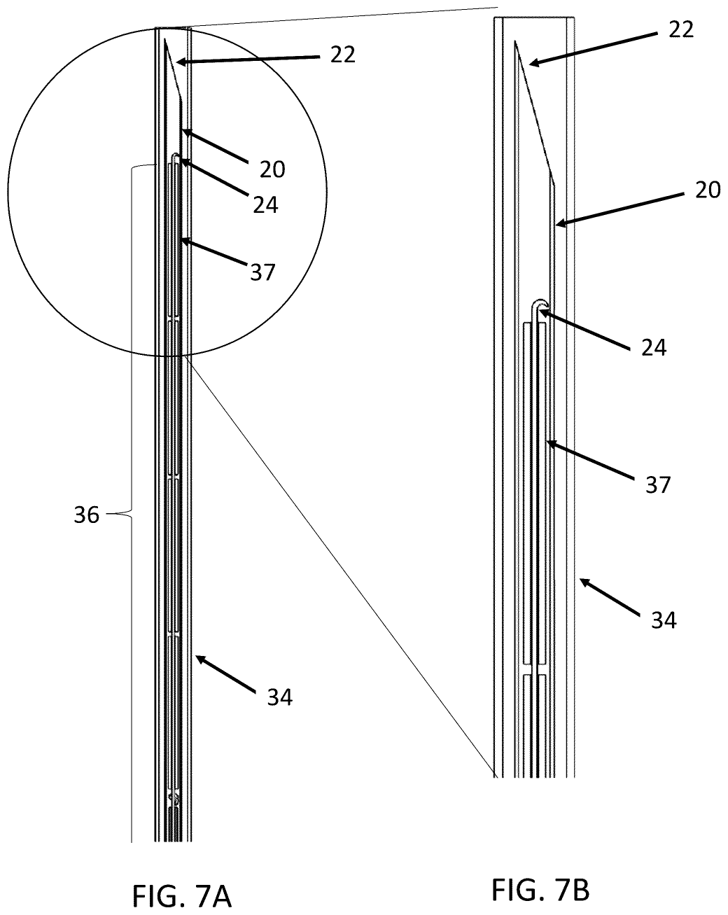

FIG. 7A shows a schematic cross section illustration of the flexible braided anchor segments 36 located in the delivery needle 20 which is concentrically positioned in the clear outer sheath 34. The suture 24 is shown extending out of the distal end of the distal anchor segment 37. In use, the needle 20 is pushed out of the clear outer sheath 34 to create the incisions, and retracted back into the clear outer sheath 34 to allow for the handle assembly 18 to be manipulated inside of the body without created unwanted cuts or incisions.

FIG. 7B shows a schematic cross section illustration of an expansion of FIG. 7A showing the flexible braided anchor segments 36 located in the delivery needle 20 which is concentrically positioned in the clear outer sheath 34.

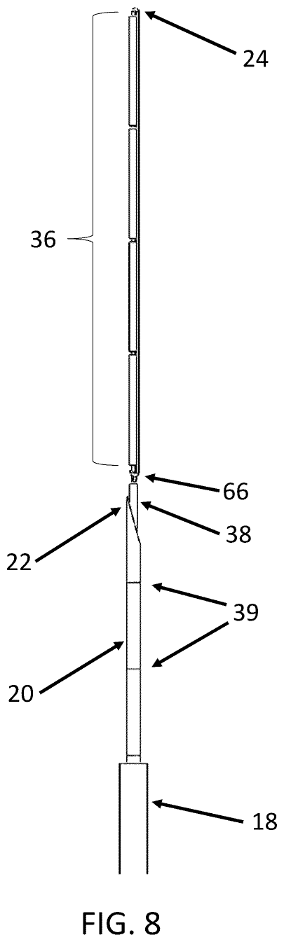

FIG. 8 shows a top view of a delivery device 16 with the delivery needle 20 extended out of the clear outer sheath 34. The drive tube 38 has pushed the plurality of braided suture segments 36 out of the needle tip 22. The suture 24 is shown being threaded through the braided suture segments 36 and looping around the distal end of the distal suture segment back to the proximal end of the proximal suture segment where the suture forms a slip knot 66.

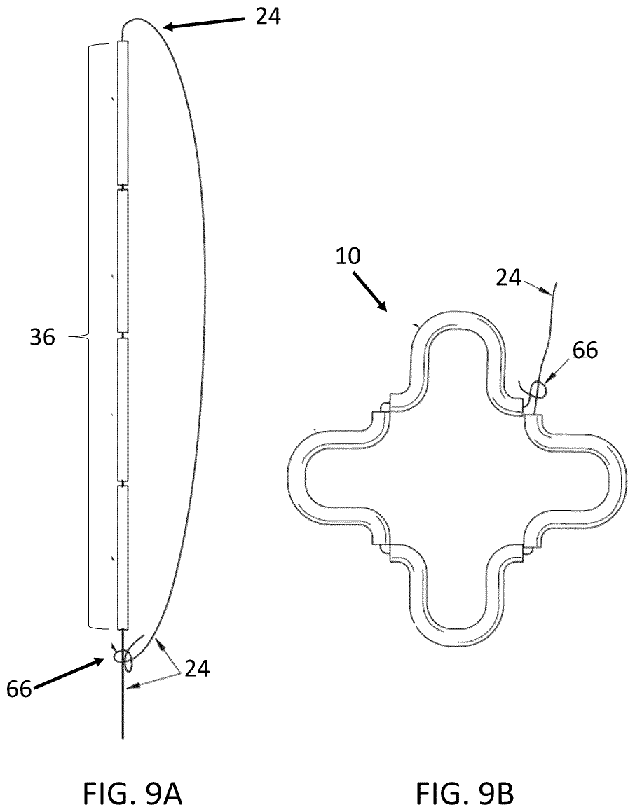

FIG. 9A shows a schematic drawing of the plurality of braided suture segments 36 with the suture 24 looping back to be formed in a slip knot 66. In this embodiment, the series of braided suture segments are strung on a suture with a slip knot 66 being positioned after the final braided suture segment on the suture 24. In this embodiment, a slip knot 66 is positioned proximal to the fourth braided suture segment 36. In this embodiment, the slip knot is positioned following the final braided suture segment threaded onto the suture. In some embodiments, the plurality of suture segments may include two, three, four, five, six, seven, eight, nine, or 10, or more suture segments.

FIG. 9B shows a schematic drawing of the plurality of braided suture segments 36 after the suture 24 has been pulled and the plurality of braided suture segments 36 has been deformed into the anchor assembly 10. As shown in FIG. 4C, above, when the suture 24 is pulled tight once it has been implanted into the organ, the suture 24 is pulled toward the slip knot 66 and becomes deformed. The deformed braided suture segments 36 become the anchor assembly 10.

In other embodiments, a slip knot or other locking mechanism may be positioned proximally to any segments of an assembly of braided suture segments. That is, a slip knot 66 or other locking mechanism may be positioned proximal to any of suture segments.

FIG. 9B shows a shows a schematic illustration of an assembly of braided anchor segments as they are deployed on the distal side of a tissue. In this non-exclusive embodiment, the assembly of braided suture segments is only anchored to the tissue at where the slip knot is pulled taut. In this embodiment, the assembly of braided anchor segments has a large surface area that enables a large securing surface formed against the tissue.

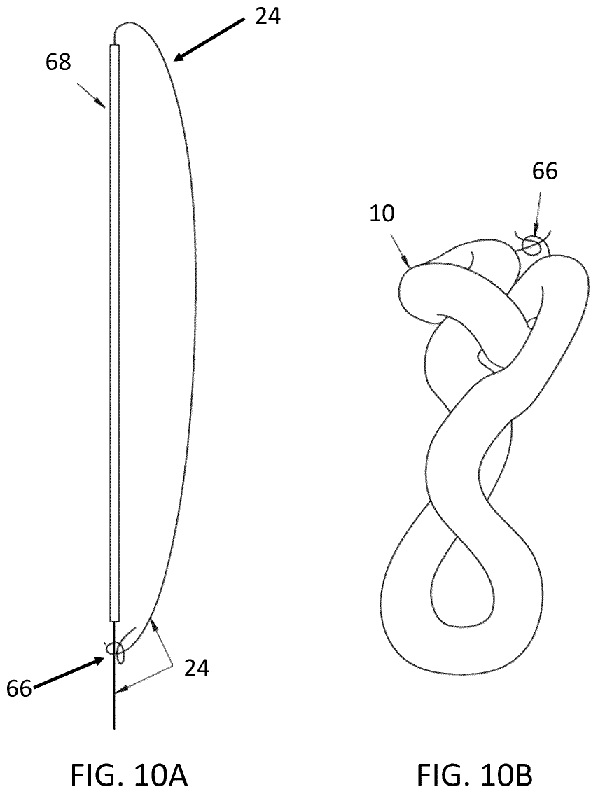

FIG. 10A shows a schematic illustration of an embodiment of a long braided suture segment 68 on a suture 24. In this embodiment the long braided suture segment 68 may be between 10 mm and 70 mm. In some embodiments, the at least one braided suture segment may be the long braided suture segment 68. In this embodiment, a slip knot 66 is positioned proximal to the long braided suture segment 68. In this embodiment, the long braided anchor 68 segment has a large surface area that enables a large securing surface with only one hole formed in the tissue. This may be useful in fixing tissue of one organ to another organ and holding it in place while other procedures are being undertaken, such as placing a stent between the two organs.

FIG. 10B shows a schematic illustration of a long braided anchor assembly 10 (e.g., deformed long braided suture segment 68) segment as it is deployed on the distal side of a tissue, such as an organ wall. In this embodiment, the deformed long braided anchor segment 68 is only anchored to the tissue at where the slip knot is pulled taut. In this embodiment, the long braided anchor segment 10 has a large surface area that enables a large securing surface with only one hole formed in the tissue. This may be useful in fixing tissue of one organ to another organ and holding it in place while other procedures are being undertaken, such as placing a stent between the two organs.

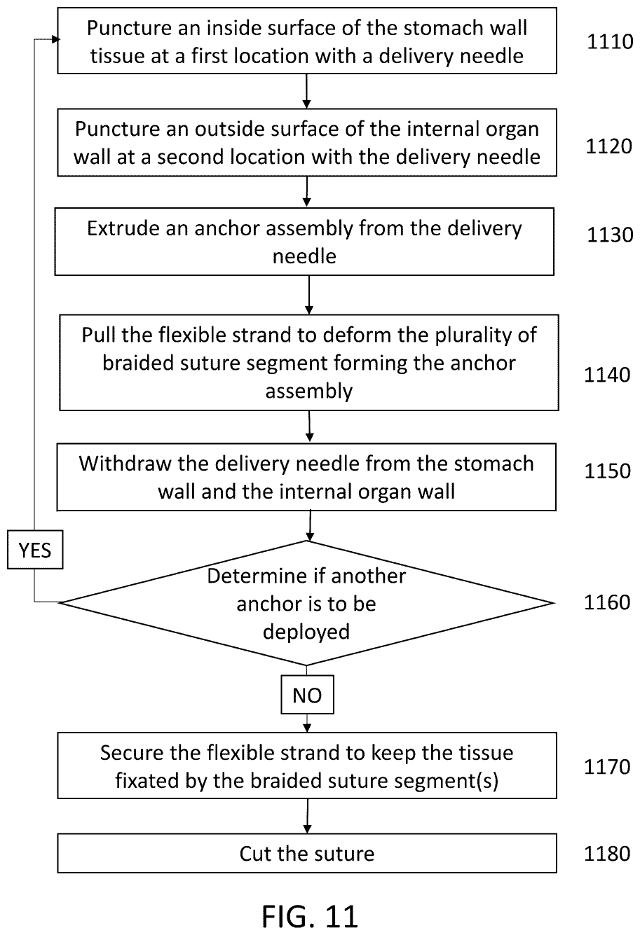

FIG. 11 shows steps of an embodiment of a method for implanting braided anchor segments connected via a single flexible strand (e.g., suture) for tissue fixation.
At 1110, puncture an inside surface of the stomach wall tissue at a first location with a delivery needle. The needle having a needle tip punctures the tissue from a proximal side of the tissue (e.g., stomach wall) to a distal side of the tissue. The needle is at a distal end of an outer tube, and the outer tube is coupled to a handle assembly.
At 1120, puncture an outside surface of the internal organ wall at a second location with the delivery needle. The needle having a needle tip punctures the tissue from a proximal side of the tissue (e.g., outside surface of the organ wall) to a distal side of the tissue. In some embodiments, the process of puncturing the outside surface of the internal organ wall can be performed continuously following the puncturing of the inside surface of the stomach wall. In some embodiments, the process of puncturing the outside surface of the internal organ wall may not be performed continuously following the puncturing of the inside surface of the stomach wall. That is, there may be some process that is engaged in between puncturing the stomach wall and the internal organ wall.
At 1130, extrude an anchor assembly from the delivery needle. The braided suture segment is slidably coupled to a flexible strand, from the delivery needle. The braided suture segment is extruded from the needle on the distal side of the tissue. The braided suture segment is pushout distally out of the needle by the first drive tube.
At 1140, pull the flexible strand to deform the plurality of braided suture segment forming the anchor assembly. As a result of the flexible strand being pulled tight, the first braided suture segment is deformed into a first braided anchor segment. The anchor assembly is configured to anchor an outside surface of the stomach wall to the outside surface of the internal organ wall, and prevent the anchor assembly from being pulled thru internal organ wall to the stomach wall.
At 1150, withdraw the delivery needle from the tissue. The delivery needle is withdrawn from the tissue leaving behind the braided anchor segment on the distal side of the tissue. When the delivery needle is retracted, the anchor assembly is left on the distal side of the tissue, (e.g., organ wall) to fix the two tissues together.
At 1160, determine if another anchor is to be deployed. A determination is made as to whether an additional braided anchor segment is required to fixate the tissue. If it is determined that an additional braided anchor segment is necessary to fixate the tissue, then the method returns to step 1110 to initiate placing an additional braided anchor segment on the distal side of the tissue. If it is determined that an additional braided anchor segment is not necessary to fixate the tissue, then the method advances to step 1170 to secure the flexible strand.
At 1170, secure the flexible strand to keep the tissue fixated by the braided suture segment(s). The flexible strand (e.g., suture) is secured by engaging a locking mechanism (e.g., cinch) with the suture to lock the suture and prevent the fixated tissue from pulling apart.
At 1180, cut the suture. The suture (e.g., flexible strand) is cut on the distal side of the locking mechanism after the flexible strand has been pulled taut and secured by the locking mechanism.

The embodiments of the invention described above are intended to be merely exemplary; numerous variations and modifications will be apparent to those skilled in the art. Such variations and modifications are intended to be within the scope of the present invention as defined by any of the appended claims.

What is claimed is:

1. An anchor assembly for fixation of a stomach wall to an internal organ wall, comprising:
a flexible strand; and
at least one braided anchor segment slidably coupled to the flexible strand, the flexible strand passing through a bore hole that traverses from a proximal end to a distal end of the at least one braided anchor segment;
wherein the at least one braided anchor segment is cinched forming an anchor assembly to provide tissue fixation.

2. The assembly of claim 1, wherein the at least one braided anchor segment comprises a straight braided suture segment that has been deformed by application of tension on the flexible strand.

3. The assembly of claim 1, wherein:
the at least one braided anchor segment comprises a plurality of braided anchor segments; and
the plurality of braided anchor segments is slidably coupled to the flexible strand, the flexible strand passing through a bore hole that traverses from a proximal end to a distal end of each of the plurality of the braided anchor segments such that the proximal end of the first braided anchor segment is adjacent to a distal end of a second braided anchor segment.

4. The assembly of claim 1, wherein the flexible strand comprises a suture.

5. The assembly of claim 4, wherein the suture comprises a braided polyester suture.

6. The assembly of claim 3, wherein each of the plurality of braided anchor segments comprise standard braided polyester suture that is cut into predetermined lengths.

7. The assembly of claim 3, wherein the cinched plurality of braided anchor segments has a purse string effect on the plurality of braided anchor segments such that the plurality of flexible braided anchor segments is drawn together to provide tissue fixation.

8. A method for implanting an anchor assembly connected via a single flexible strand for fixation of a stomach wall to an internal organ wall, comprising:
puncturing an inside surface of the stomach wall tissue at a first location with a delivery needle;
puncturing an outside surface of the internal organ wall at a second location with the delivery needle;
extruding an anchor assembly from the delivery needle, the anchor assembly comprising:
the single flexible strand; and
a plurality of braided anchor segments slidably coupled to the single flexible strand, the single flexible strand passing through a bore hole that traverses from a proximal end to a distal end of each of the plurality of braided anchor segments such that the proximal end of a first braided anchor segment is adjacent to a distal end of a second braided anchor segment;
pulling the flexible strand a first time to deform the plurality of braided anchor segments forming the anchor assembly, the anchor assembly configured to: anchor an outside surface of the stomach wall to the outside surface of the internal organ wall; and prevent the anchor assembly from being pulled thru internal organ wall to the stomach wall;
withdrawing the delivery needle from the stomach wall and the internal organ wall;
securing the flexible strand to keep the stomach wall anchored to the internal organ wall fixated by the anchor assembly; and
cutting the flexible strand, the flexible strand is cut a distance away from the proximal end of a final braided anchor segment of the plurality of the braided anchor segments.

9. The method of claim 8, wherein the extruding the anchor assembly is accomplished by advancing a drive tube to push the plurality of braided anchor segments out of the delivery needle.

10. The method of claim 8, wherein:
the plurality of braided anchor segments comprises a slip knot placed around the flexible strand at the proximal end of the proximal braided anchor segment; and
the securing the flexible strand comprises locking the flexible strand in place using a locking mechanism comprising at least one of a tag, a barb, a cinch, a toggled needle, or a distel knot.

11. The method of claim 8, wherein the securing the flexible strand comprises a locking device or an overhand knot that can be run down to a fixation site.

12. The method of claim 8, wherein puncturing the outside surface of the internal organ wall at the second location with the delivery needle follows continuously from puncturing the inside surface of the stomach wall tissue at the first location with the same delivery needle.

13. A surgical device for implanting a flexible braided anchor assembly for affixing a stomach wall to an internal organ wall, comprising:
a handle assembly, the handle assembly having a cylindrical bore passing through the handle assembly on a center axis along a length of the handle assembly;
a delivery needle positioned in the cylindrical bore of the handle assembly, the delivery needle being hollow and having a common center line with the cylindrical bore of the handle assembly, the delivery needle comprising an outer tube having a needle tip at a distal end of the delivery needle;
at least one braided suture segment slidably coupled to a flexible strand, the flexible strand being translationally threaded through of the at least one braided suture segment, the at least one braided suture segment positioned in the delivery needle;
a first drive tube positioned in the delivery needle and having a common center line with the delivery needle, the first drive tube configured to extrude the at least one braided suture segment.

14. The surgical device of claim 13, wherein the at least one braided suture segment comprises a hollow braided suture segment.

15. The surgical device of claim 13, wherein:
the flexible strand comprises a monofilament strand;
the at least one braided anchor segment comprises a plurality of braided anchor segments;
the plurality of braided anchor segments is slidably coupled to the flexible strand, the flexible strand passing through a bore hole that traverses from a proximal end to a distal end of each of the plurality of the braided anchor segments such that the proximal end of the first braided anchor segment is adjacent to a distal end of a second braided anchor segment; and
the plurality of braided suture segments comprises a plurality of hollow braided suture segments aligned end to end having the monofilament strand threaded through the aligned plurality of hollow braided suture segments.

16. The surgical device of claim 15, wherein a column strength of the plurality of hollow braided suture segments is increased by application of at least one of heat or a cyanoacrylate coating to each of the segments of the plurality of hollow braided suture segments.

17. The surgical device of claim 15, wherein a braided suture segment of the plurality of braided suture segments has a length of between about 5 mm and about 50 mm.

18. The surgical device of claim 15, wherein each of the braided suture segments of the plurality of braided suture segments has a length of between about 5 mm and about 12 mm.

19. The surgical device of claim 16, wherein a distal end of the flexible strand threaded thru the first braided suture segment is looped around the first braided suture segment and knotted around the flexible strand between the first braided suture segment and the first drive tube.

20. The surgical device of claim 15, wherein each of the plurality of braided suture segments is treated to increase a rigidity of the plurality of braided suture segments slidably coupled to the flexible strand.

21. The surgical device of claim 20, wherein the treatment comprises:
heating each of the plurality of braided suture segments; or
applying a cyanoacrylate coating to each of the plurality of braided suture segments.

22. The surgical device of claim 13, wherein the stomach comprises a duodenum.

23. A method for implanting an anchor assembly connected via a single flexible strand through a drain tube placed from the outside of a body for fixation of a gall bladder wall to a stomach wall, comprising:
puncturing an inside surface of the gall bladder wall tissue at a first location with a delivery needle, the delivery needle being brought into the interior of the gall bladder through a drain tube placed between outside of the body and the gall bladder;
puncturing an outside surface of the stomach wall at a second location with the delivery needle;
extruding an anchor assembly from the delivery needle, the anchor assembly comprising:
the single flexible strand; and
at least one braided anchor segment slidably coupled to the single flexible strand, the single flexible strand passing through a bore hole that traverses from a proximal end to a distal end of the at least one braided anchor segment;
pulling the flexible strand a first time to deform the at least one braided anchor segment forming the anchor assembly, the anchor assembly configured to: anchor an inside surface of the stomach wall to the outside surface of the gall bladder wall; and prevent the anchor assembly from being pulled thru the stomach wall to the gall bladder wall;
withdrawing the delivery needle from the stomach wall and the gall bladder wall;
securing the flexible strand to keep the stomach wall anchored to the gall bladder wall fixated by the anchor assembly; and
cutting the flexible strand, the flexible strand is cut a distance away from the proximal end of the at least one braided anchor segments.
